(19) United States
(12) Patent Application Publication
(10) Pub. No.: US 2025/0302613 A1
(43) Pub. Date: Oct. 2, 2025

(54) PROSTHETIC VALVES WITH THROMBUS ANCHORING STRIPS

(71) Applicant: EDWARDS LIFESCIENCES CORPORATION, Irvine, CA (US)

(72) Inventors: Tamir S. Levi, Zikhron Yaakov (IL); Evan T. Schwartz, Huntington Beach, CA (US); Anatoly Dvorsky, Haifa (IL)

(73) Assignee: EDWARDS LIFESCIENCES CORPORATION, Irvine, CA (US)

(21) Appl. No.: 19/232,042

(22) Filed: Jun. 9, 2025

Related U.S. Application Data
(63) Continuation of application No. PCT/US2023/082844, filed on Dec. 7, 2023.
(60) Provisional application No. 63/432,734, filed on Dec. 15, 2022.

(51) Int. Cl. A61F 2/24

(52) U.S. Cl. CPC A61F 2/2418; A61F 2220/0008; A61F 2250/0037

(57) ABSTRACT

The present disclosure relates to prosthetic valves configured to maintain thrombotic tissue that may form in low-flow or recirculation zones thereof from traveling downstream the valve. Such prosthetic valves can include a frame movable between a radially compressed state and a radially expanded state, a strip circumferentially disposed about an inner surface of the frame, and a plurality of leaflets coupled to the strip, wherein the strip is configured to allow tissue adherence to an internal surface thereof. The prosthetic valve can optionally include an inner skirt disposed between the frame and the strip.

### CROSS-REFERENCE TO RELATED APPLICATIONS

This application is a continuation of PCT Application No. PCT/US2023/082844, filed Dec. 7, 2023, which claims the benefit of U.S. Provisional Application No. 63/432,734, filed Dec. 15, 2022, the entire contents of each of which are incorporated by reference herein.

### FIELD

The present invention relates to prosthetic valves that include strips disposed internally to the frames of the valves and configured to allow adherence of thrombus or other tissue thereto.

### BACKGROUND

Native heart valves, such as the aortic, pulmonary and mitral valves, function to assure adequate directional flow from, and to, the heart, and between the heart's chambers, to supply blood to the whole cardiovascular system. Various valvular diseases can render the valves ineffective and require replacement with artificial valves. Surgical procedures can be performed to repair or replace a heart valve. Conventional surgically implantable prosthetic valve typically include a leaflet assembly mounted within a relatively rigid support frame or ring. Components of the prosthetic valve are usually assembled with one or more biocompatible fabrics, and a fabric-covered sewing ring is provided around the valve for suturing to the tissue of the native leaflet.

Since surgeries are prone to an abundance of clinical complications, alternative less invasive techniques of delivering a prosthetic heart valve over a catheter and implanting it over the native malfunctioning valve have been developed over the years. Different types of prosthetic heart valves are known to date, including balloon expandable valve, self-expandable valves and mechanically-expandable valves.

Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, toward the native malfunctioning valve. Once the prosthetic valve is properly positioned at the desired site of implantation, it can be expanded against the surrounding anatomy, such as an annulus of a native valve, and the delivery assembly can be retrieved thereafter.

### SUMMARY

Most expandable prosthetic valve include flexible leaflets attached to expandable frames thereof, wherein the leaflets are configured to transition between closed and open states, so as to regulate flow of blood through the prosthetic valves. In some cases, regions of the prosthetic valve might be subjected to blood stasis, low flow, or recirculation zones between the leaflets and the frame. Stagnant or otherwise disturbed pools of blood behind the leaflets can cause thrombus formations, loosely attached to an inner skirt covering the inner surface of the valve's frame and facing the leaflets. Since most of the conventional inner skirts are thromboresistant, or at least not formed to encourage tissue adherence thereto, such thrombotic deposits may occasionally detach from the skirt and travel downstream, posing a risk of occluding narrower portions of the vasculature.

According to some aspects of the disclosure, there is provided a prosthetic valve comprising a frame, a strip circumferentially disposed about an inner surface of the frame, and a valvular structure coupled to the strip. The frame is movable between a radially compressed state and a radially expanded state. The strip comprises a strip internal surface facing a central axis of the prosthetic valve. The valvular structure comprises a plurality of leaflets extending inward from the strip internal surface.

In some examples, the strip is configured to allow tissue adherence to the strip internal surface.

According to some aspects of the disclosure, there is provided a prosthetic valve comprising a frame, an inner skirt coupled to an inner surface of the frame, a strip coupled to an internal surface of the skirt, and a plurality of leaflets coupled to the strip internal surface. The frame is movable between a radially compressed state and a radially expanded state. The leaflets are configured to regulate flow through the prosthetic valve. The strip comprises a strip internal surface.

In some examples, the inner skirt is configured to allow formation of neointimal tissue that does not exceed an average thickness threshold.

In some examples, the strip is configured to encourage adherence of tissue of any thickness to the strip internal surface.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1A is a perspective view of an exemplary prosthetic valve.

FIG. 1B is a perspective view of the prosthetic valve of FIG. 1A, without the outer skirt.

FIG. 1C is a perspective view of a frame of the prosthetic valve of FIGS. 1A-B.

FIG. 2 is a perspective view of an example of a valvular structure that includes three leaflets.

FIG. 3 shows an exemplary delivery apparatus carrying a balloon expandable prosthetic valve.

FIG. 4 is a cross-sectional view of an exemplary prosthetic valve, showing thrombotic dislodgements from an inner skirt thereof.

FIGS. 5A and 5B are cross-sectional views of exemplary prosthetic valves that include s trip to which the leaflets are attached.

FIG. 6 is a cross-sectional view of an exemplary prosthetic valve, showing tissue formation over the strip.

FIG. 7 is a partial view of a portion of an outer skirt having a textured external surface.

FIG. 8 is a partial view of a portion of a strip having a textured strip internal surface.

FIG. 9 is a perspective view of an exemplary prosthetic valve with a strip having an undulating shape.

FIGS. 10A-10C are partial views of exemplary strips having uniform and non-uniform heights.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

It should be understood that the disclosed examples can be adapted to deliver inflatable balloons, and in some implementations, to deliver and implant prosthetic devices expandable by such inflatable balloons, to and/or in any of the native annuluses of the heart (e.g., the aortic, pulmonary, mitral, and tricuspid annuluses), and can be used with any of various delivery approaches (e.g., retrograde, antegrade, transseptal, transventricular, transatrial, etc.).

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid unduc clutter from having too many reference numbers and lead lines on a particular drawing, some components will be introduced via one or more drawings and not explicitly identified in every subsequent drawing that contains that component.

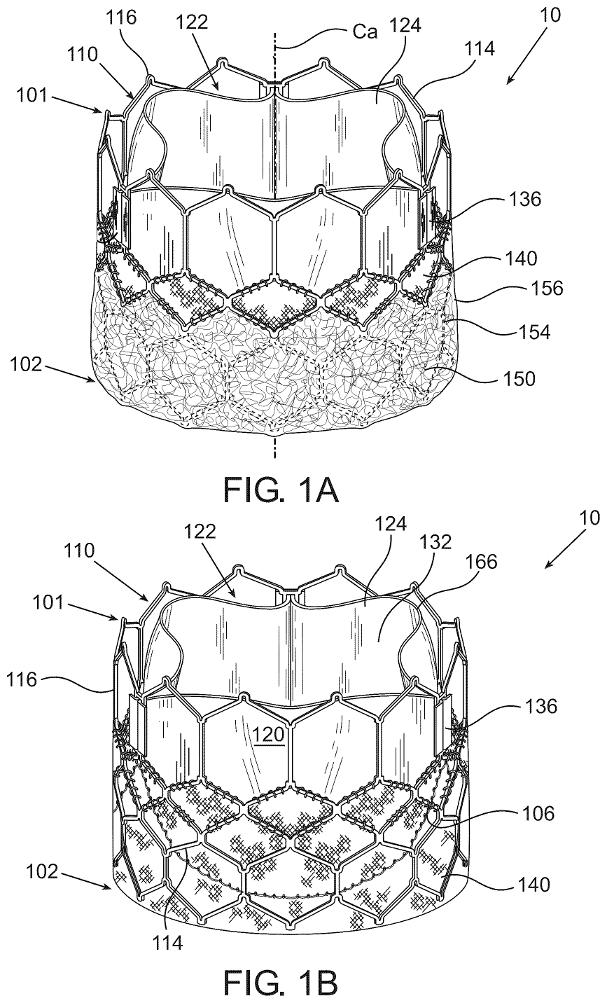

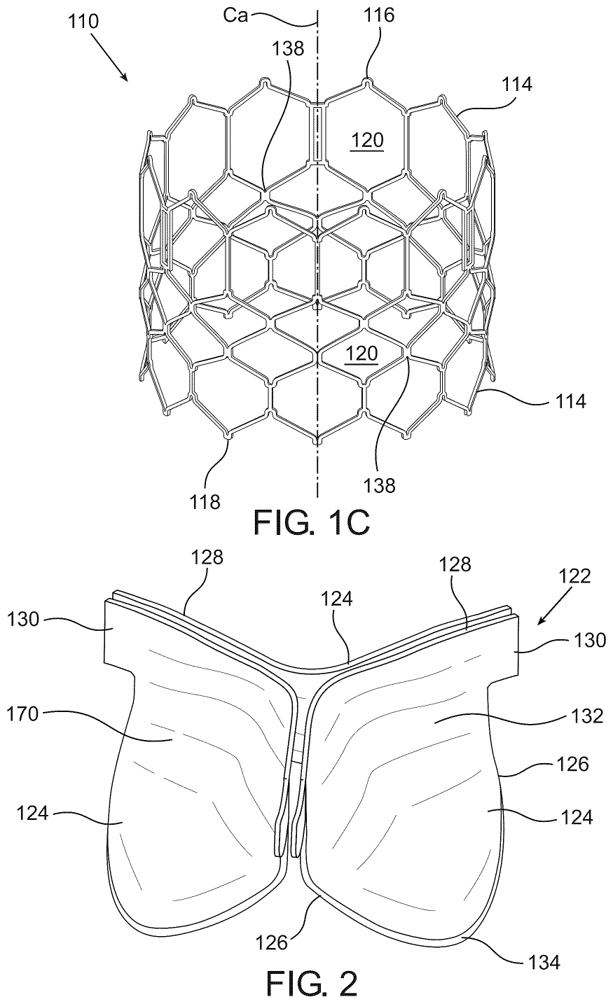

FIGS. 1A and 1B show perspective views of an exemplary prosthetic valve 10, with and without an outer skirt 150 surrounding the frame 110, respectively. FIG. 1C shows the frame 110 without any other soft components attached thereto. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valve can be crimped on or retained by an implant delivery apparatus (not shown) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (e.g., prosthetic valve 10, 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/085,947 and 63/209,904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 1A-1C show an example of a prosthetic valve 10, which can be a balloon expandable valve or any other type of valve, illustrated in an expanded state. The prosthetic valve 10 can comprise an outflow end 101 and an inflow end 102. In some instances, the outflow end 101 is the proximal end of the prosthetic valve 10, and the inflow end 102 is the distal end of the prosthetic valve 10. Alternatively, depending for example on the delivery approach of the valve, the outflow end can be the distal end of the prosthetic valve, and the inflow end can be the distal end of the proximal valve.

The term “outflow”, as used herein, refers to a region of the prosthetic valve through which the blood flows through and out of the prosthetic valve 10.

The term “inflow”, as used herein, refers to a region of the prosthetic valve through which the blood flows into the prosthetic valve 10.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The terms “longitudinal” and “axial”, as used herein, refer to an axis extending in the proximal and distal directions, unless otherwise expressly defined.

The prosthetic valve 10 comprises an annular frame 110 movable between a radially compressed configuration and a radially expanded configuration, and a valvular structure 122 mounted within the frame 110. The frame 110 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 110 can be crimped to a radially compressed state on a balloon catheter, and then expanded inside a patient by an inflatable balloon or equivalent expansion mechanism. Alternatively or additionally, the frame 110 can be made of shape-memory materials such as, but not limited to, nickel titanium alloy (e.g., Nitinol). When constructed of a shape-memory material, the frame 110 can be crimped to a radially compressed state and restrained in the compressed state by insertion into a shaft or equivalent mechanism of a delivery apparatus.

In the example illustrated in FIGS. 1A-IC, the frame 110 is an annular, stent-like structure comprising a plurality of intersecting struts 114. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 114 may be any elongated member or portion of the frame 110. The frame 110 can include a plurality of strut rungs that can collectively define one or more rows of cells 120. The frame 110 can have a cylindrical or substantially cylindrical shape having a constant diameter from the inflow end 102 to the outflow end 101 as shown, or the frame can vary in diameter along the height of the frame, as disclosed in U.S. Pat. No. 9,155,619, which is incorporated herein by reference.

The end portions of the struts 114 are forming apices 116 at the outflow end 101 and apices 118 at the inflow end 102. The struts 114 can intersect at additional junctions 138 formed between the outflow apices 116 and the inflow apices 118. The junctions 138 can be equally or unequally spaced apart from each other, and/or from the apices 116, 118, between the outflow end 101 and the inflow end 102.

The struts 114 can include a plurality of angled struts and vertical or axial struts. FIGS. 1A-1C show an exemplary prosthetic valve 10 that can be representative of, but is not limited to, a balloon expandable prosthetic valve. The frame 110 of the prosthetic valve 10 illustrated in FIG. 1C comprises rungs of angled struts and axial struts disposed between some of the rungs of the angled struts. In such implementations of the frame, the struts can be pivotable or bendable relative to each other, so as to permit frame expansion or compression. For example, the frame 110 can be formed from a single piece of material, such as a metal tube, via various processes such as, but not limited to, laser cutting, electroforming, and/or physical vapor deposition, while retaining the ability to collapse/expand radially in the absence of hinges and like.

A valvular structure 122, shown also for example in FIG. 2, can include a plurality of leaflets 124 (e.g., three leaflets), positioned at least partially within the frame 110, and configured to regulate flow of blood through the prosthetic valve 10 from the inflow end 102 to the outflow end 101. While three leaflets 124 arranged to collapse in a tricuspid arrangement, are shown in the example illustrated in FIGS. 1A-1B and 2, it will be clear that a prosthetic valve 10 can include any other number of leaflets 124. Adjacent leaflets 124 can be arranged together to form commissures 136 that are coupled (directly or indirectly) to respective portions of the frame 110, thereby securing at least a portion of the valvular structure 122 to the frame 110. The leaflets 124 can be made from, in whole or part, biological material (e.g., pericardium), bio-compatible synthetic materials, or other such materials. Further details regarding transcatheter prosthetic valves, including the manner in which the valvular structures 122 can be coupled to the frame 110 of the prosthetic valve 10, can be found, for example, in U.S. Pat. Nos. 6,730,118, 7,393,360, 7,510,575, 7,993,394, 8,652,202, and 11,135,056, all of which are incorporated herein by reference in their entireties.

As shown for example in FIG. 2, three separate leaflets 124 can collectively define the valvular structure 122 in some cases. Each leaflet 124 can have a rounded cusp edge 126 opposite a free edge 128, and a pair of generally oppositely-directed tabs 130 separating the cusp edge 126 and the free edge 128. The cusp edge 126 in such cases forms a single scallop. Each leaflet 124 further comprises an inner surface (not annotated), defined as the surface facing the valve central axis Ca (indicated in FIG. 1A), and an outer surface (not annotated), opposite thereto so as to face the frame 110.

When such leaflets 124 are coupled to the frame and to each other, the lower edge of the resulting valvular structure 122 desirably has an undulating, curved scalloped shape. By forming the leaflets with this scalloped geometry, stresses on the leaflets 124 are reduced which, in turn, improves durability of the prosthetic valve. Moreover, by virtue of the scalloped shape, folds and ripples at the belly of each leaflet, which can cause early calcification in those areas, can be eliminated or at least minimized. The scalloped geometry also reduces the amount of tissue material used to form the valvular structure, thereby allowing a smaller, more even crimped profile at the inflow end of the valve.

The leaflets 124 define a non-planar coaptation plane (not annotated) when their free edges 128 co-apt with each other to seal blood flow through the prosthetic valve 10. Leaflets 124 can be secured to one another at their tabs 130 to form commissures 136 of the valvular structure 122, which can be secured, directly or indirectly, to structural elements connected to the frame 110 or integrally formed as portions thereof, such as commissure posts, commissure windows, and the like. When secured to two other leaflets 124 to form valvular structure 122, the cusp edges 126 of the leaflets 124 collectively form the scalloped line 106 of the valvular structure 122. Each leaflet 124 comprises a leaflet body 132, defined between the line of attachment of the leaflet to the frame, for example along scalloped line 106, and the free edge 128. The leaflet body 132 defines the movable portion of the leaflet 124, free to move toward the frame 110 in an open state of the valvular structure 122, and toward central axis Ca to co-apt with other leaflets 124 in a closed state of the valvular structure 122. The lowest or distal-most end of each leaflet 124 is at cusp edge midpoint 134, which in turn also defines the lowest or distal-most region of scalloped line 106.

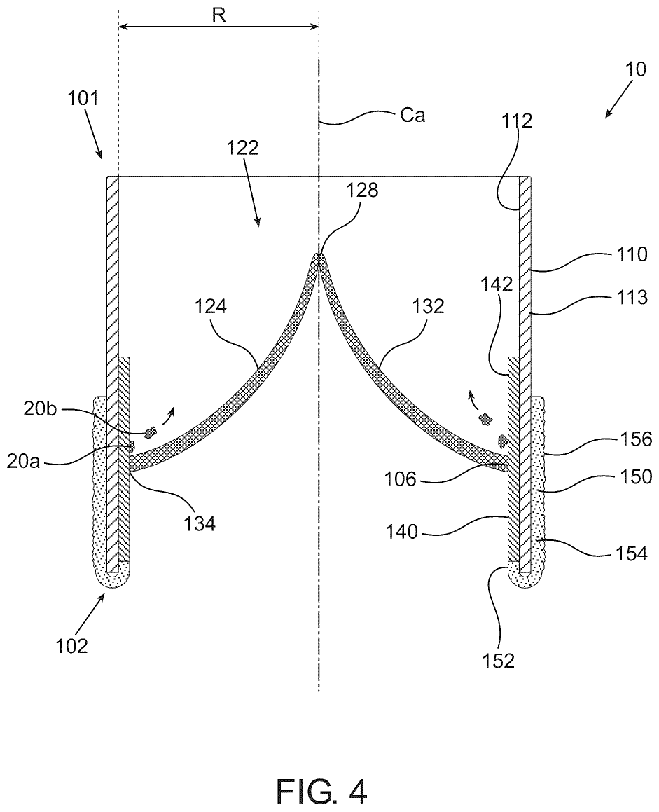

In some examples, the prosthetic valve 10 can comprise at least one skirt or scaling member. FIGS. 1A-1B show an example of a prosthetic valve 10 that includes an inner skirt 140, which can be secured to the inner surface 112 (annotated, for example, in FIG. 4) of the frame 110. Such an inner skirt 140 can be configured to function, for example, as a scaling member to prevent or decrease perivalvular leakage. An inner skirt 140 can further function as an anchoring region for valvular structure 122 to the frame 110, and/or function to protect the leaflets 124 against damage which may be caused by contact with the frame 110, for example during valve crimping or during working cycles of the prosthetic valve 10. FIG. 1B shows an inner skirt 140 disposed around and attached to the inner surface 112 of frame 110, wherein the valvular structure 122 is sutured to the inner skirt 140 along scalloped line 106. The inner skirt 140 can be coupled to the frame 110 via sutures or another form of coupler.

The prosthetic valve 10 can comprise, in some examples, an outer skirt 150 mounted on the outer surface 113 (annotated, for example, in FIG. 4) of frame 110, configure to function, for example, as a sealing member retained between the frame 110 and the surrounding tissue of the native annulus against which the prosthetic valve is mounted, thereby reducing risk of paravalvular leakage (PVL) past the prosthetic valve 10. The outer skirt 150 can be coupled to the frame 110 via sutures or another form of coupler.

Any of the inner skirt 140 and/or outer skirt 150 can be made of various suitable biocompatible materials, such as, but not limited to, various synthetic materials (e.g., PET) or natural tissue (e.g. pericardial tissue). In some cases, the inner skirt 140 can be formed of a single sheet of material that extends continuously around the inner surface 112 of frame 110. In some cases, the outer skirt 150 can be formed of a single sheet of material that extends continuously around the outer surface 113 of frame 110.

The outer skirt 150 can define an internal surface (not annotated) facing and optionally contacting the outer surface 113 of the frame 110, and an opposite external surface 156 facing away from the frame 110, toward the surrounding anatomy when implanted in a patient's body. In some examples, as illustrated for example in a cross-sectional view of prosthetic valve 10 in FIG. 4, the outer skirt can include an outer portion 154 extending over at least a portion of the outer surface 113 of frame 110, and fold over the frame 110 along the inflow end 102 to further include an inner portion 152 extending over a portion of the inner surface 112 of frame 110, extending over a limited height between the inflow end 102 and the inner skirt 140, for example. In such cases, the external surface 156 of the outer skirt 150 can be defined only over the outer portion 154, and not the inner portion 152. Such configurations, in which the outer skirt 150 covers inflow apices 118, can advantageously provide an atraumatic inflow end 102 of the prosthetic valve 10, preventing the inflow apices 118 from accidentally engaging or snagging portions of the delivery apparatus 200 during advancement through the patient's vasculature to the site of implantation.

While an outer skirt 150 is illustrated throughout some of the drawings of the current specification, such as FIGS. 4-6, to fold over inflow apices 118 so as to include both an inner portion 152 and an outer portion 154, it is to be understood that this configuration is shown by way of illustration and not limitation, and that an outer skirt 150 can similarly extend only over the outer surface 113 of the frame 110, such that it includes the outer portion 154 and does not necessarily define an inner portion 152.

In some implementations, the inner skirt 140 is configured to be relatively thromboresistant. For example, the inner skirt 140 can include at least one or more of a polytetrafluorocthylene (PTFE), an ultra-high molecular weight polyethlene (UHMWPE), or a coated thermoplastic polyurethane (TPU). The polytetrafluoroethylene (PTFE) may comprise expanded polytetrafluorocthylene (cPTFE) in some examples as desired. Various other thromboresistant materials may be utilized as desired. In some examples, a thromboresistant coating may be utilized with the inner skirt 140. For example, a coating of PTFE or ePTFE may be applied to material of the inner skirt 140, which may comprise polyethylene terephthalate (PET) or another form of material. The underlying material of the inner skirt 140 in such examples, may be a material that allows for tissue ingrowth, such as PET, yet is coated with a thromboresistant coating. In some examples, the material of the inner skirt 140 may be fabricated to be thromboresistant. For example, a knit pattern of the material of the inner skirt 140 may be configured to be thromboresistant, with micropatterns or other forms of knit patterns. A knit PTFE fabric may be utilized in some examples. The material of the inner skirt 140 can include a smooth texture to be thromboresistant and inhibit tissue growth. Combinations of such features may be utilized to result in a thromboresistant inner skirt 140.

In some examples, the outer skirt 150 is configured to allow tissue ingrowth, at least along outer portion 154. In some examples, the outer skirt 150 can include polyethylene terephthalate (PET) or another form of material that is configured to allow tissue ingrowth. In some examples, a coating may be utilized with the outer skirt 150 that is configured to allow tissue ingrowth. For example, a porous coating or other form of coating may be applied to a thromboresistant material such as ultra-high molecular weight polyethlene (UHMWPE) to allow for tissue ingrowth. The underlying material of the outer skirt 150 in such examples, may be a material that inhibits tissue ingrowth, yet is coated with a coating that allows tissue ingrowth, at least over outer portion 154. In some examples, the material of the outer skirt 150 may be fabricated to allow tissue ingrowth. For example, a knit pattern of the material of the outer skirt 150 may be configured to allow tissue ingrowth, with a large knit pattern or other forms of knit patterns. A knit PET fabric may be utilized in some examples. In some examples, the outer skirt 150 may include yarns (e.g., textured yarns) extending radially outward. The material of the outer skirt 150 may include a porous texture to allow for tissue ingrowth. Combinations of such features may be utilized to result in an outer skirt 150 that allows for tissue ingrowth. It is to be understood that for implementations of outer skirts 150 that include both an inner portion 152 and an outer portion 154, any of the examples described hereinabove with respect to encouraging tissue ingrowth may be applied to the outer portion 154, while the inner portion 152 may or may not be configured to encourage tissue ingrowth.

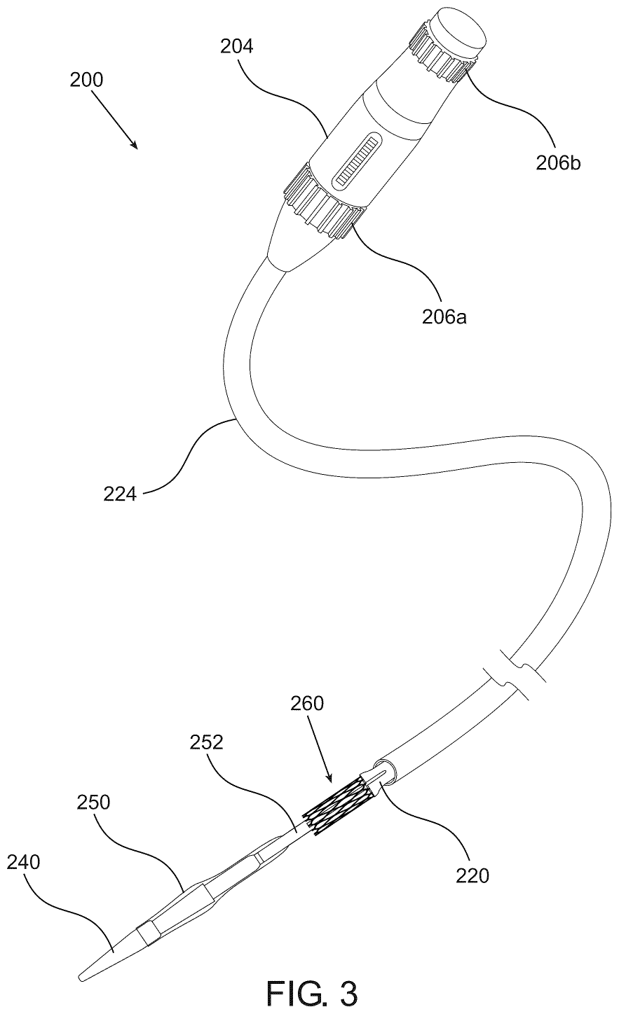

FIG. 3 illustrate a delivery apparatus 200, according to an exemplary configuration, adapted to deliver a balloon expandable prosthetic valve 260 described herein (e.g., prosthetic valve 10 or 100). It should be understood that the delivery apparatus 200 can be used to implant prosthetic devices other than prosthetic valves, such as stents or grafts.

The delivery apparatus 200 includes a handle 204 and a balloon catheter 252 having an inflatable balloon 250 mounted on its distal end. The prosthetic valve 260 can be carried in a crimped state over the balloon catheter 252. Optionally, an outer delivery shaft 224 can concentrically extend over the balloon catheter 252, and a push shaft 220 can be disposed over the balloon catheter 252, optionally between the balloon catheter 252 and the outer delivery shaft 224.

The outer delivery shaft 224, the push shaft 220, and the balloon catheter 252, can be configured to be axially movable relative to each other. For example, a proximally oriented movement of the outer delivery shaft 224 relative to the balloon catheter 252, or a distally oriented movement of the balloon catheter 252 relative to the outer delivery shaft 224, can expose the prosthetic valve 260 from the outer delivery shaft 224. The delivery apparatus 200 can further include a nosecone 240 carried by a nosecone shaft (hidden from view in FIG. 3) extending through a lumen of the balloon catheter 252.

The proximal ends of the balloon catheter 252, the outer delivery shaft 224, the push shaft 220, and optionally the nosecone shaft, can be coupled to the handle 204. During delivery of the prosthetic valve 260, the handle 204 can be maneuvered by an operator (e.g., a clinician or a surgeon) to axially advance or retract components of the delivery apparatus 200, such as the nosecone shaft, the balloon catheter 252, the outer delivery shaft 224, and/or the push shaft 220, through the patient's vasculature, as well as to inflate the balloon 250 mounted on the balloon catheter 252, so as to expand the prosthetic valve 260, and to deflate the balloon 250 and retract the delivery apparatus 200 once the prosthetic valve 260 is mounted in the implantation site.

The handle 204 can include a steering mechanism configured to adjust the curvature of the distal end portion of the delivery apparatus 200. In the illustrated example, the handle 204 includes an adjustment member, such as the illustrated rotatable knob 206a, which in turn is operatively coupled to the proximal end portion of a pull wire. The pull wire can extend distally from the handle 204 through the outer delivery shaft 224 and has a distal end portion affixed to the outer delivery shaft 224 at or near the distal end of the outer delivery shaft 224. Rotating the knob 206a can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein. The handle 204 can further include an adjustment mechanism including an adjustment member, such as the illustrated rotatable knob 206b. The adjustment mechanism can be configured to adjust the axial position of the push shaft 220 relative to the balloon catheter.

The prosthetic valve 260 can be carried by the delivery apparatus 200 during delivery in a crimped state, and expanded by balloon inflation to secure it in a native heart valve annulus. In an exemplary implantation procedure, the prosthetic valve 260 is initially crimped over the balloon catheter 252, proximal to the inflatable balloon 250. Because prosthetic valve 260 is crimped at a location different from the location of balloon 250, prosthetic valve 260 can be crimped to a lower profile than would be possible if it was crimped on top of balloon 250. This lower profile permits the clinician to more easily navigate the delivery apparatus 200 (including crimped prosthetic valve 260) through a patient's vasculature to the treatment location. The lower profile of the crimped prosthetic valve is particularly helpful when navigating through portions of the patient's vasculature which are particularly narrow, such as the iliac artery.

The balloon 250 can be secured to balloon catheter 252 at its balloon proximal end, and to either the balloon catheter 252 or the nosecone 240 at its distal end. The distal end portion of the push shaft 220 is positioned proximal to the outflow end (e.g., outflow end 101) of the prosthetic valve 260.

When reaching the site of implantation, and prior to balloon inflation, the push shaft 220 is advanced distally, allowing its distal end portion to contact and push against the outflow end of prosthetic valve 260, pushing the valve 260 distally therewith. The distal end of push shaft 220 is dimensioned to engage with the outflow end of the prosthetic valve 260 in a crimped configuration of the valve. In some implementations, the distal end portion of the push shaft 220 can be flared radially outward, to terminate at a wider-diameter that can contact the prosthetic valve 260 in its crimped state. Push shaft 220 can then be advanced distally, pushing the prosthetic valve 260 therewith, until the crimped prosthetic valve 260 is disposed around the balloon 250, at which point the balloon 250 can be inflated to radially expand the prosthetic valve 260. Once the prosthetic valve 260 is expanded to its functional diameter within a native annulus, the balloon 250 can be deflated, and the delivery apparatus 200 can be retrieved from the patient's body.

In particular implementations, the delivery apparatus 200 with the prosthetic valve 260 assembled thereon, can be packaged in a sterile package that can be supplied to end users for storage and eventual use. In particular implementations, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the prosthetic valve 260 and the delivery apparatus 200, can be free of any liquid. Methods for treating tissue leaflets for dry storage are disclosed in U.S. Pat. Nos. 8,007,992 and 8,357,387, both of which documents are incorporated herein by reference.

FIG. 4 shows a cross sectional view of prosthetic valve 10. In some cases, blood stagnation or low flow regions can occur between leaflets 124 and inner skirt 140, particularly at the lowest or distal-most regions along the scalloped line 106, in the vicinity of cusp edge midpoint 134. Such “pockets” of flow stagnation behind the leaflets can cause platelets to adhere to the internal surface 142 of the inner skirt 140, until thrombus formation along the inner skirt 140. Since a conventional inner skirt 140 is usually designed to resist tissue ingrowth, for example by being made of thromboresistant materials and/or by including relatively smooth internal surfaces 142, adhesion of thrombotic formations 20 to the inner skirt 140 is relatively loose, which is of critical concern because, if dislodged, such thrombus can create an embolus that may reach important systemic organs.

FIG. 4 schematically illustrates several exemplary thrombus formations at various optional stages, with thrombus 20a shown to be formed over and loosely attached to the internal surface 142 of inner skirt 140, for example within a “flow pocket” in the vicinity of cusp edge midpoint 134, and another thrombus 20b detached from the inner skirt 140, free to migrate further downstream, eventually causing blood clots or blockage in other portions of the artery.

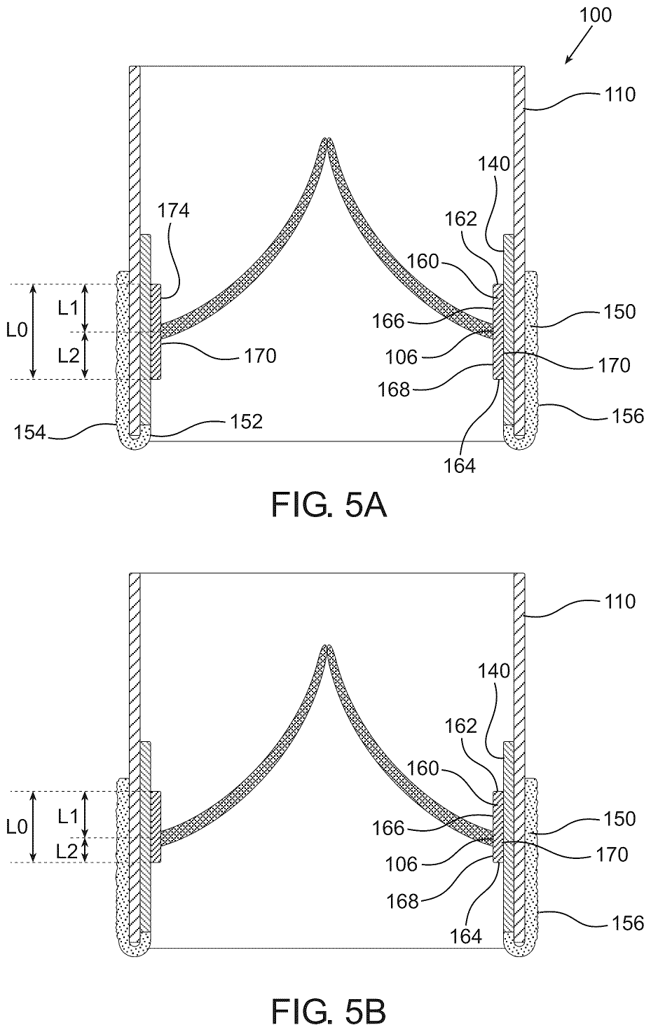

FIG. 5A shows a cross-sectional view of an exemplary implementation of a prosthetic valve 100. Prosthetic valve 100 can be structurally and functionally similar to any example described above with respect to prosthetic valve 10, except that while the leaflets 124 of prosthetic valve 10 are directly attached (e.g., sutured) along the scalloped line 106 to the inner skirt 140, prosthetic valve 100 further comprises a strip 160 disposed internally to the frame 110, and more specifically, disposed about the inner surface 112 of the frame 110. The strip 160 can be disposed around and attached to the internal surface 142 of inner skirt 140, while the leaflets 124 are attached (e.g., sutured) along the scalloped line 106 to the strip 160, such that the leaflets 124 extend inward (i.e., toward central axis Ca) from an internal surface 174 of the strip 160. All other components and features of prosthetic valve 100 can be similar to the same components, with the same component numerals, and features thereof, described above with respect to prosthetic valve 10, and in the interest of brevity will not be described further.

Various exemplary implementations for prosthetic valves 100 can be referred to, throughout the specification, with superscripts, for case of explanation of features that refer to such exemplary implementations. It is to be understood, however, that any reference to structural or functional features of any apparatus, assembly or component, without a superscript, refer to these features being commonly shared by all specific exemplary implementations that can be also indicated by superscripts. In contrast, features emphasized with respect to an exemplary implementation of any apparatus, assembly or component, including prosthetic valves 100, referred to with a superscript, may be optionally shared by some but not necessarily all other exemplary implementations.

The strip 160 can be disposed around the entire circumference of inner skirt 140, and more particularly, the internal surface 142 of inner skirt 140. The strip 160 can be coupled to the inner skirt 140 via bonding, stitching, or another form of coupling. The strip 160 comprises a proximal edge 162 and a distal edge 164, and defines a strip height L0, parallel to central axis Ca, between the proximal edge 162 and the distal edge 164. The strip height L0 can be less than the height of the inner skirt 140 (not annotated separately), such that the proximal edge 162 of the strip 160 can be distal to the proximal edge of the inner skirt 140, and such that the distal edge 164 of the strip 160 can be proximal to the distal edge of the inner skirt 140.

The leaflets 124 are attached to the strip 160 along scalloped line 106 at a position distal to the proximal edge 162 of the strip 160, such that the strip 160 comprises a proximal portion 166 extending along a proximal portion height L1 between the scalloped line 106 and the proximal edge 162 of the strip. In some examples, the scalloped line 106 is positioned between the proximal edge 162 and the distal edge 164 of the strip, and in particular, proximal to the distal edge 164, such that the strip 160 further comprises a distal portion 168 extending along a distal portion height L2 between the scalloped line 106 and the proximal edge 162 of the strip. In some examples, the strip 160 does not include a distal portion 168, such that the scalloped line 106 is formed along the distal edge 164 of the strip, in which case the proximal portion height L1 is also equal to the total strip height L0. When the strip 160 includes both a proximal portion 166 and a distal portion 168, their combined heights L1+L2 together define the total strip height L0.

The strip 160, which can be also referred to as a thrombus anchoring strip 160, comprises a strip internal surface 174, defined as the surface facing toward central axis Ca and away from frame 110. The strip 160, and in particular, its internal surface 174, is configured to encourage tissue ingrowth and/or tissue adherence thereto, such that any thrombus formed along the strip 160 will remain attached thereto, thereby reducing risk of thrombus dislodgment from the strip 160, such as in the vicinity of cusp edge midpoint 134 or other regions along the scalloped line 106. The proximal portion 166 is configured to encourage tissue ingrowth thereover and/or tissue adherence thereto at a region bound between an inner surface of the leaflet 124 (facing the frame 110, inner skirt 140 and strip 160) and the strip internal surface 174, such as low-flow, recirculation zone or stagnation pockets formed between the lower portion of the leaflet 124 and the strip 160, above the scalloped line 106.

When present, the distal portion 168 can be configured to similarly encourage tissue ingrowth thereover and/or tissue adherence thereto at a region bound between the leaflet 124 and the strip 160, such as low-flow, recirculation zone or stagnation pockets formed between the lower portion of an outer surface of the leaflet 124 (facing central axis Ca), extending over at least a portion of the distal portion 168 of the strip, below the scalloped line 106. While flow disturbances that can encourage thrombosis are more prominent within the “flow pockets” in the vicinity of cusp edge midpoints 134 above the scalloped line 106, flow disturbances can occasionally occur also below the leaflets 124 in the vicinity of and distal to the scalloped line 106 and/or cusp edge midpoints 134, optionally to a lesser extent, yet sufficient to encourage thrombosis in such regions as well, in which case it may be of advantage for the strip 160 to include a distal portion 168 in addition to the proximal portion 166.

When strip 160 includes both a proximal portion 166 and a distal portion 168, their heights can be similar or different from each other. FIG. 5A shows an exemplary implementation in which the proximal portion height L1 is substantially equal to the distal portion height L2. The term substantially equal to, as used herein, refer to being within a range of ±10% of the target value.

In some examples, the distal portion length L2 is equal to the proximal portion length L1. FIG. 5A illustrates an exemplary implementation of a prosthetic valve 100 provided with a strip 160 defining a distal portion height L2 substantially equal to the proximal portion height L1. In some examples, the distal portion length L2 is less than the proximal portion length L1. FIG. 5B illustrates an exemplary implementation of a prosthetic valve 100 provided with the strip 160 similar to the example shown in FIG. 5A, except that the valve 100 shown in FIG. 5B has the strip 160 having a distal portion height L2 which is less than the proximal portion height L1. In some examples, the proximal portion height L1 is at least twice as great as the distal portion height L2 (i.e., L1>2L2), such that L2 constitutes at least two thirds of the strip height L0 (i.e., L1>2/3L) while L1 constitutes no more than a third of the strip height (L2≤1/3L). It is to be understood that any other ratio of L1 to L2 is contemplated, such as 3:2, 5:3, 7:3, and the like.

In some examples, the strip 160 is configured to allow tissue ingrowth or tissue adherence thereto. In some examples, the strip 160 can include polyethylene terephthalate (PET) or another form of material that is configured to allow tissue ingrowth or tissue adherence thereto. In some examples, a coating may be utilized with the strip 160 that is configured to allow tissue ingrowth or tissue adherence thereto. For example, a porous coating or other form of coating may be applied to a thromboresistant material such as ultra-high molecular weight polyethlene (UHMWPE) to allow for tissue ingrowth or tissue adherence thereto. The underlying material of the strip 160 in such examples, may be a material that inhibits tissue ingrowth, yet is coated with a coating that allows tissue ingrowth or tissue adherence thereto, at least along the proximal portion 166. In some examples, the material of the strip 160 may be fabricated to allow tissue ingrowth or tissue adherence thereto. For example, a knit pattern of the material of the strip 160 may be configured to allow tissue ingrowth or adherence, with a large knit pattern or other forms of knit patterns formed along the strip internal surface 174. A knit PET fabric may be utilized in some examples. In some examples, the strip 160 may include yarns (e.g., textured yarns) extending radially inward. The material of the strip 160 may include a porous texture to allow for tissue ingrowth. Combinations of such features may be utilized to result in a strip 160 that allows for tissue ingrowth or tissue adherence thereto.

The term “tissue adherence”, as used herein with respect to strip 160, refers both to tissue ingrowth directly within strip 160 or over the strip internal surface 174, as well as to the strip's ability to capture and maintain attachment with thrombus that may be formed in close proximity thereto, such as within the “floc pocket” bound between the strip 160 and leaflet 124, or along the outer surface of the leaflet 124. For example, a thrombus that may initiate formation within the “flow pocket” due to stagnation or flow recirculation, may freely float within the pocket and be contacted by a textures surface (such as inwardly extending yarns) of the strip, upon which the thrombus will adhere to the strip internal surface 174, and remain attached thereto in a manner that prevents, or at least reduces the likelihood of, spontaneous dislodgment therefrom.

The strip 160 is configured to prevent spontaneous detachment of tissue adhered thereto, for a minimal period of time, after which spontaneous dislodgment becomes less likely. The term “spontaneous detachment”, as used herein, refers to detachment from the strip 160 when subjected to normal physiological and flow conditions at the site of implantation (or within an experimental setup that mimics such conditions). In some examples, the strip 160 is configured to prevent spontaneous detachment of tissue adhered thereto (either when implanted in a patient or in an experimental setup that mimics the flow conditions of a physiological implantation site) for a period of at least one month. In some examples, the strip 160 is configured to prevent spontaneous detachment of tissue adhered thereto for a period of at least six months. In some examples, the strip 160 is configured to prevent spontaneous detachment of tissue adhered thereto for a period of at least one year.

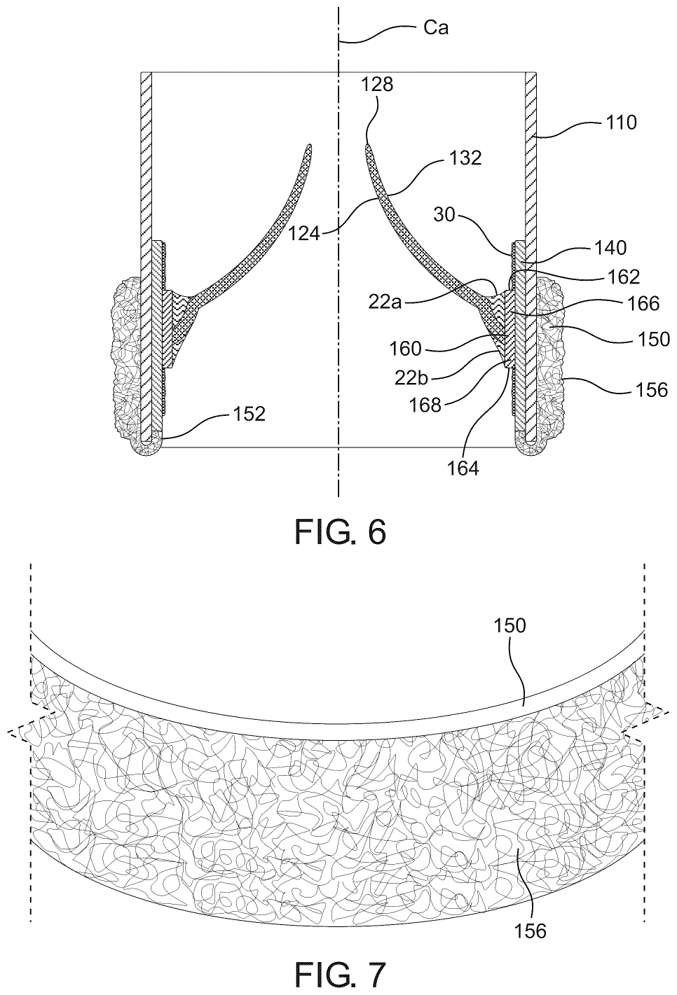

FIG. 6 shows a cross sectional view of an exemplary prosthetic valve 100 with tissue ingrowth and/or thrombus adherence to different portions thereof, representing a potential state of the valve 100 after residing within the implantation site for a certain period of time. As mentioned above, the outer skirt 150, and more specifically, an outer portion 154 thereof, can be configured to allow tissue ingrowth in some examples, which may result in a bulkier or thicker formation of the outer skirt 150 over time, as schematically shown in FIG. 6.

Thrombus 22a that may have formed along the proximal portion 166 of strip 160, is shown to remain firmly attached to the strip 160 due to the materials and/or textures provided by strip 160 to allow long-term adherence of tissue thereto, as described above. Another thrombus 22b is shown in FIG. 6 formed along the distal portion 168 of the strip, similarly demonstrated to remain attached to the strip 160 with reduce risk of dislodging therefrom.

In some cases, the portion of the leaflet 124 facing the proximal portion 166 of strip 160 and the thrombus 22a formed thereon, is also adhered to the opposite side of thrombus 22a, such that the thrombus 22a is bound between, and attached to, both the proximal portion 166 of strip 160 and the corresponding lower portion of the outer surface of leaflet 124. This may, in turn, inhibit motility of the lower portion of leaflet 124 attached to thrombus 22a to a certain extent, such that the portion of leaflet body 132 free to move between the open and closed states of the valvular structure 122 is the portion above thrombus 22a.

FIGS. 5A-5B shows the valvular structure 122 in a closed state, wherein the leaflets 124 are shown to co-apt with each other to prevent backflow through the main flow channel defined between the leaflets 124. The leaflets 124 can be dimensioned to allow their free edges 128 to move along the inner radius R (indicated, for example, in FIG. 4) between the open state, in which their free edges 128 can move closer to the inner surface 112 of the frame 110, and the closed state, moving toward each other and the central axis Ca. When a portion of the leaflet 124 is adhered to thrombus 22a, full coaptation may not be necessarily achievable. Nevertheless, it has been shown in a series of experiments that a limited degree of backflow may be allowed in the closed state, without adversely affecting the performance of the prosthetic valve.

In some cases, thrombus 22a is adhered to the proximal portion 166 of strip 160 and the corresponding lower portion of the outer surface of leaflet 124 in a manner that can allow full coaptation between the leaflets 124, yet limit their opening, such that the resulting orifice area through which blood can flow between the leaflets in their open state is less than the orifice area in an un-constricted state.

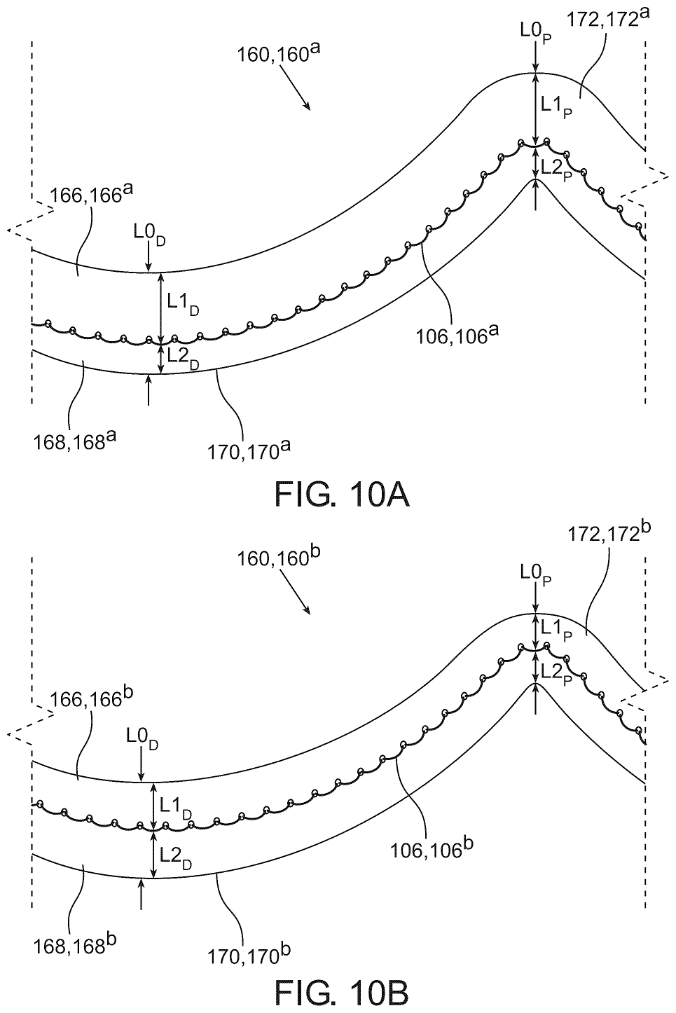

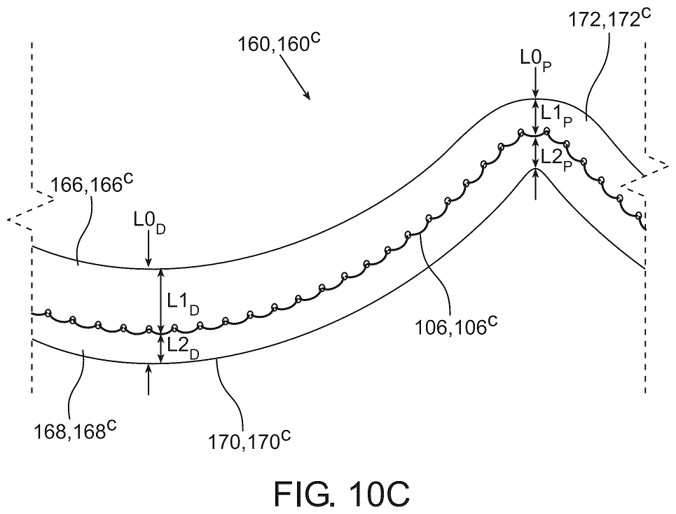

Thus, the proximal portion height L1 of strip 160 can be designed such that even when a portion of leaflet 124 adhered to a thrombus 22a along a portion extending from the scalloped line 106, optionally from the cusp edge midpoint 134, along a length equal to the length L1, the amount of backflow through the valvular structure 122 will still not exceed the tolerable threshold beyond which the performance of the valve 100 can be adversely affected, and/or the flow of blood through a potentially narrower orifice area between the leaflets will still be sufficient to maintain adequate valve functioning. In some examples, the proximal portion height L1 is equal to or less than 20% of internal radius R of prosthetic valve 100 in the expanded state. In some examples, the proximal portion height L1 is equal to or less than 10% of internal radius R of prosthetic valve 100 in the expanded state. In some implementations, a strip 160 can have a non-uniform proximal portion height L1 along its circumference, as will be described in greater detail with respect to FIG. 10A-10C herein. In such cases, any reference to a proximal portion height L1 not exceeding a certain percentage of the radius R can refer to a maximal proximal portion height L1, which can be at the position of cusp edge midpoint 134, also referred to as inflow end portion 170 of the strip 160, as will be described in greater detail with respect to FIG. 9 herein.

In some examples, the inner skirt 140 is not entirely thromboresistant, but is rather configured to allow neointimal tissue formation over its internal surface 142. This can be achieved by a porous internal surface 142 configured to allow neointimal tissue layer formation therealong. The term “neointimal tissue”, referred to herein as a tissue layer 30 (shown in FIG. 6) that can form along internal surface 142 of inner skirt 140, refers to a thin layer of tissue having an average thickness (in the radial direction) of no more than 200 μm (or another average thickness threshold). The internal surface 142 can have an adequate degree of porosity configured to encourage ingrowth and/or adherence of a neointimal tissue layer that does not exceed the desired thickness. Thus, it is to be understood that even when the inner skirt 140 is configured to encourage neointimal tissue formation thereover, it differs from the strip 160 in that the skirt 140 is configured to allow formation of a tissue layer that has an average thickness of 200 μm (or another average thickness threshold), while the strip 160 is configured to allow adherence of thrombus or other tissue layers of any thickness, extending thrombotic formations having an average thickness exceeding 200 μm (or another average thickness threshold), or even exceeding 0.5 mm.

In some examples, the strip 160 and the inner skirt 140 can be made from different materials. In some examples, the strip internal surface 174 and the internal surface 142 of the inner skirt 140 can have different textures. In some examples, the strip internal surface 174 can include filaments extending radially inward, while internal surface 142 can be relatively smooth and/or porous. In some examples, both the strip internal surface 174 and the internal surface 142 of the inner skirt 140 are porous surfaces, but with differently dimensioned pores, such that the pores of strip internal surface 174 can be relatively larger to allow adherence of thrombus of any size thereto, while the pores of the inner skirt 140 can be relatively narrower to encourage neointimal tissue formation that will not exceed an average thickness of about 200 μm (or another average thickness threshold).

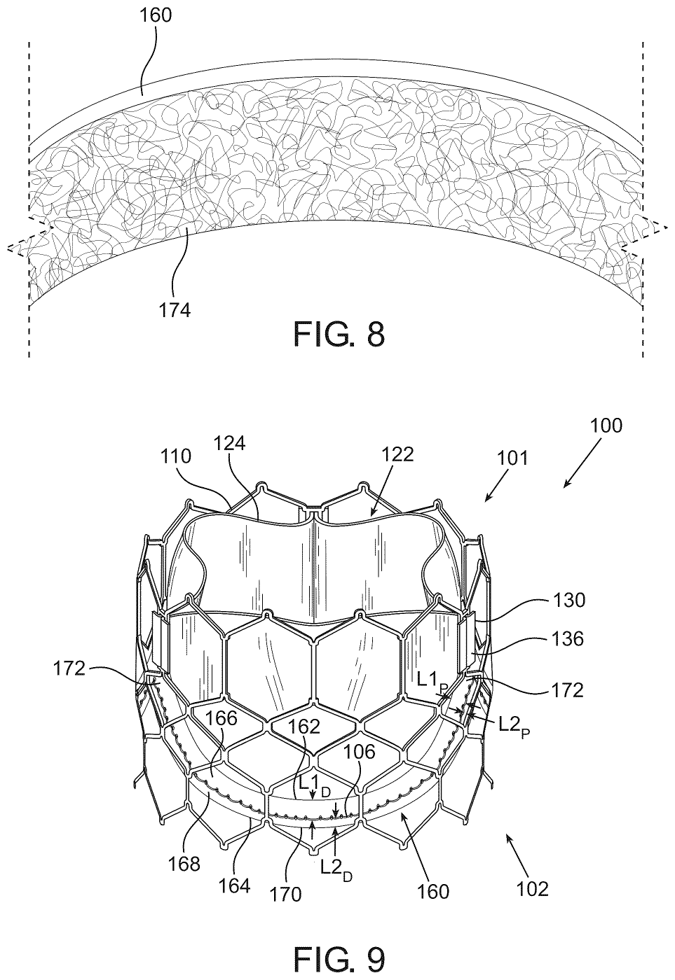

FIGS. 7-8 illustrate partial isolated perspective views of soft components of the prosthetic valve configured to allow tissue ingrowth or tissue adherence thereto. FIG. 7 shows a portion of an exemplary outer skirt 150, and in particular, an outer portion 154 thereof. In some examples, the external surface 156 of outer skirt 150 can include filaments extending therefrom. The filaments may further allow tissue ingrowth within the outer skirt 150. In some examples, only a portion of external surface 156 can include filaments.

FIG. 8 shows a portion of an exemplary strip 160. In some examples, the strip internal surface 174 can include filaments extending therefrom. The filaments may further allow tissue ingrowth within the strip 160 and/or enhance adherence of thrombus 22 to the strip 160. In some examples, only a portion of strip internal surface 174 can include filaments, such as the proximal portion 166.

FIG. 9 shows a view in perspective of an exemplary prosthetic valve 100, with the inner skirt 140 and outer skirt 150 removed from view to expose the strip 160. Strip 160 can be formed of a single continuous piece of material extending along the entire inner circumference of frame 110 and/or inner skirt 140, or it can be formed of separate components, such as three sub-components, each attached to the cusp edge 126 of one of three corresponding leaflets 124. The strip 160 can have a scalloped configuration when mounted to the inner skirt 140, which can generally track the shape of scalloped line 106 along which the leaflets 124 are attached to the strip 160. The strip 160 can thus define inflow end portions 170, which are the distal ends of the strip 160 corresponding to the positions of cusp edge midpoints 134, closer to the inflow end 102 of the valve, and outflow end portions 172, which are the proximal ends of strip 160 adjacent commissures 136, closer to the outflow end 101 of the valve. The cross-sectional views of FIGS. 5A-6 can be representative of views taken across inflow end portions 170 of strips 160.

Any of the height L0, L1, L2 can be uniform or non-uniform along the circumference of the strip, and particularly between the inflow end portions 170 and the outflow end portions 172. FIGS. 10A-10C show partial views of exemplary strips that can be used in lieu of the strip 160 shown in FIG. 9. The heights L0_{D}, L1_{D}, and L2_{D}, denote the strip height, proximal portion height, and distal portion height, respectively, at the inflow end portion 170 of the strip. The heights L0_{P}, L1_{P}, and L2_{P}, denote the strip height, proximal portion height, and distal portion height, respectively, at the outflow end portion 172 of the strip 160.

FIG. 10A shows a strip 160ª, which is an exemplary implementation of strip 160, wherein while a strip 160 of prosthetic valve 100 can have uniform or non-uniform strip height L0, uniform or non-uniform proximal portion height L1, and uniform or non-uniform distal portion height L2, the strip 160^{a} has uniform heights L1, L2 and L0. For example, as shown in FIG. 10A, strip 160^{a} is shown to have a uniform proximal portion height L1 such that L1_{D} is substantially equal to L1_{P}, and a uniform distal portion height L2 such that L2_{P} is substantially equal to L2_{P}, which will result in a uniform strip height L0 such that L0_{P} is substantially equal to L0_{P}.

The uniform proximal portion height L1 can be equal to or different from the uniform distal portion height L2. For example, the strip 160^{a} illustrated in FIG. 10A is shown to have a uniform proximal portion height L1 which is greater than the uniform distal portion height L2. However, since the heights L1 and L2 of strip 160^{a} are uniform, the same ratio of L1 to L2 is maintained between the inflow end portions 170 and outflow end portions 172. In such examples, the curvatures of the proximal edge 162 and the distal edge 164 of the strip 160^{a} can be similar to each other and to the curvature of the scalloped line 106.

In some examples, the strip height L0 and/or the proximal height L1 can be greater at the inflow end portions 170 and narrower at the outflow end portions. Such configurations can be advantageous when flow stagnation or other flow disturbances are mostly prominent in the distal-most “pockets” formed between the leaflets and the strip 160 and/or inner skirt 140, in the vicinity of cusp edge midpoints 134, increasing the likelihood of thrombosis in this regions, compared with the likelihood of thrombus formation at more proximal regions closer to the outflow end 101, or the expected size of thrombotic tissue formed closer to the outflow end portions 172 of the strip 160.

FIG. 10B shows strip 160^{b}, which is an exemplary implementation of strip 160, wherein while a strip 160 of prosthetic valve 100 can have uniform or non-uniform strip height L0, uniform or non-uniform proximal portion height L1, and uniform or non-uniform distal portion height L2, the strip 160^{b} has non-uniform heights L1, L2 and L0. For example, as shown in FIG. 10B, strip 160^{b} is shown to have a non-uniform proximal portion height L1 narrowing from a greater height L1 at the inflow end portions 170 to a smaller height L1 at the outflow end portions 172 (i.e., L1_{D}>L1_{P}), and a non-uniform uniform distal portion height L2 narrowing from a greater height L2 at the inflow end portions 170 to a smaller height L2 at the outflow end portions 172 (i.e., L2_{D}>L2_{P}), which will result in a non-uniform strip height L0 narrowing from the inflow end portions 170 to the outflow end portions 172 (i.e., L0_{D}>L0_{P}).

The non-uniform proximal portion height L1 can be equal to or different from the non-uniform distal portion height L2. For example, the strip 160^{b} illustrated in FIG. 10B is shown to have a non-uniform proximal portion height L1 which is greater than the non-uniform distal portion height L2. However, while the heights L1 and L2 of strip 160^{b} are non-uniform, the ratio of L1 to L2 can optionally remain uniform between the inflow end portions 170 and outflow end portions 172.

FIG. 10C shows a strip 160^{c}, which is an exemplary implementation of strip 160, wherein while a strip 160 can have uniform or non-uniform strip height L0, uniform or non-uniform proximal portion height L1, and uniform or non-uniform distal portion height L2, the strip 160^{c} of prosthetic valve 100^{c} has a non-uniform height L1 and a uniform height L2. For example, as shown in FIG. 10C, strip 160^{c} is shown to have a non-uniform proximal portion height L1 narrowing from a greater height L1_{D} at the inflow end portions 170 to a narrower height L1_{P} at the outflow end portions 172, uniform distal portion height L2 between its inflow end portions 170 and outflow end portions 172 (i.e., L2_{D}=L2_{P}), which results in a non-uniform strip height L0 narrowing from the inflow end portions 170 to the outflow end portions 172 (i.e., L0_{D}>L0_{P}), as well as a non-uniform ratio of L1 to L2.

While FIGS. 9-10C illustrate exemplary implementations of strips 160 that include both proximal 166 and distal portions 168, it is to be understood that a strip 160 that includes a proximal portion 166 without a distal portion 168, can be similarly provided with a uniform or non-uniform proximal portion height L1.

In some implementations, the outer skirt 150 is configured to be bioresorbable. A bioresorbable outer skirt 150 may be constructed of a bioresorbable fabric or other bioresorbable materials. Upon implantation, the bioresorbable outer skirt 150 may be configured to dissolve, and may be removed due to the dissolving and/or may be replaced by tissue in its place. Such a feature may result in tissue formation around the implanted prosthetic valve 100 that may enhance sealing against the surrounding anatomy.

While various examples of prosthetic valve 100 are described above and illustrated throughout some of the drawings to include an inner skirt 140, it is to be understood that in some examples, a prosthetic valve 100 can be devoid of an inner skirt. In such examples, the strip 160 can be disposed about and in direct contact with the inner surface 112 of the frame 110, and can be directly coupled to the frame 110, such as by stitching or using other forms of coupler, and/or by being coupled, through opening of cells 120, to the outer skirt 150.

While various examples of prosthetic valve 100 are described above and illustrated throughout some of the drawings to include an outer skirt 150 configured to allow tissue ingrowth, it is to be understood that in some examples, the outer skirt 150 does not include features that encourage such ingrowth.

While various examples of prosthetic valve 100 are described above and illustrated throughout some of the drawings to include an outer skirt 150, it is to be understood that in some examples, a prosthetic valve 100 can be devoid of an outer skirt.

### SOME EXAMPLES OF THE DISCLOSED IMPLEMENTATIONS

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A prosthetic valve, comprising:
a frame movable between a radially compressed state and a radially expanded state;
a strip circumferentially disposed about an inner surface of the frame, the strip comprising a strip internal surface facing a central axis of the prosthetic valve, wherein the strip is configured to allow tissue adherence to the strip internal surface; and
a valvular structure coupled to the strip and comprising a plurality of leaflets extending inward from the strip internal surface.

Example 2. The prosthetic valve of any example herein, particularly example 1, wherein the valvular structure is coupled to the strip along a scalloped line, and wherein the strip comprises at least one inflow end portion and at least one outflow end portion.

Example 3. The prosthetic valve of any example herein, particularly example 2, wherein the strip comprises a proximal portion extending between the scalloped line and a proximal edge of the strip, wherein the proximal portion defines a proximal portion height.

Example 4. The prosthetic valve of any example herein, particularly example 3, wherein the proximal portion height at the at least one inflow end portion is equal to or less than 20% of a radius of the prosthetic valve in the radially expanded state.

Example 5. The prosthetic valve of any example herein, particularly example 4, wherein the proximal portion height the at least one inflow end portion is equal to or less than 10% of the radius of the prosthetic valve in the radially expanded state.

Example 6. The prosthetic valve of any example herein, particularly any one of examples 3 to 5, wherein the proximal portion height is non-uniform between the at least one inflow end portion and the at least one outflow end portion.

Example 7. The prosthetic valve of any example herein, particularly example 6, wherein the proximal portion height at the at least one outflow end portion is smaller than the proximal portion height at the at least one inflow end portion.

Example 8. The prosthetic valve of any example herein, particularly any one of examples 2 to 7, wherein the strip further comprises a distal portion extending between the scalloped line and a distal edge of the strip, wherein the distal portion defines a distal portion height.

Example 9. The prosthetic valve of any example herein, particularly example 8, wherein the proximal portion height is greater than the distal portion height at the at least one inflow end portion.

Example 10. The prosthetic valve of any example herein, particularly example 9, wherein the proximal portion height is at least twice as great as the distal portion height at the at least one inflow end portion.

Example 11. The prosthetic valve of any example herein, particularly any one of examples 8 to 10, wherein the distal portion height is non-uniform between the at least one inflow end portion and the at least one outflow end portion.

Example 12. The prosthetic valve of any example herein, particularly example 11, wherein the distal portion height at the at least one outflow end portion is smaller than the proximal portion height at the at least one inflow end portion.

Example 13. The prosthetic valve of any example herein, particularly any one of examples 2 to 12, wherein the plurality of leaflets comprises three leaflets, and wherein the at least one inflow end portion of the strip comprises three inflow end portions.

Example 14. The prosthetic valve of any example herein, particularly any one of examples 1 to 13, wherein the plurality of leaflets are made from biological material.

Example 15. The prosthetic valve of any example herein, particularly example 14, wherein the biological material comprises pericardium.

Example 16. The prosthetic valve of any example herein, particularly any one of examples 1 to 15, wherein the valvular structure is coupled to the strip by bonding or stitching.

Example 17. The prosthetic valve of any example herein, particularly any one of examples 1 to 16, wherein the strip comprises yarns extending radially inward.

Example 18. The prosthetic valve of any example herein, particularly any one of examples 1 to 16, wherein the strip comprises polyethylene terephthalate.

Example 19. The prosthetic valve of any example herein, particularly any one of examples 1 to 16, wherein the strip comprises a knit pattern formed along the strip internal surface.

Example 20. The prosthetic valve of any example herein, particularly any one of examples 1 to 16, wherein the strip comprises a porous texture.

Example 21. The prosthetic valve of any example herein, particularly any one of examples 1 to 20, wherein the strip is configured to allow adherence of tissue exceeding an average thickness of 200 μm to the strip internal surface.

Example 22. The prosthetic valve of any example herein, particularly any one of examples 1 to 21, wherein the strip is configured to prevent spontaneous detachment of tissue adhered thereto for at least a predetermined period of time.

Example 23. The prosthetic valve of any example herein, particularly example 22, wherein the predetermined period of time is at least one month.

Example 24. The prosthetic valve of any example herein, particularly example 22, wherein the predetermined period of time is six months.

Example 25. The prosthetic valve of any example herein, particularly example 22, wherein the predetermined period of time is one year.

Example 26. The prosthetic valve of any example herein, particularly any one of examples 1 to 25, further comprising an inner skirt coupled to the inner surface of the frame, wherein the strip is coupled to an internal surface of the inner skirt.

Example 27. The prosthetic valve of any example herein, particularly example 26, wherein the strip defines a strip height which is less than a height of the inner skirt.

Example 28. The prosthetic valve of any example herein, particularly example 26 or 27, wherein the inner skirt is thromboresistant.

Example 29. The prosthetic valve of any example herein, particularly example 26 or 27, wherein the inner skirt is configured to allow neointimal tissue formation over its internal surface.

Example 30. The prosthetic valve of any example herein, particularly example 29, wherein the inner skirt is configured to allow formation of neointimal tissue that does not exceed an average thickness of 200 μm.

Example 31. The prosthetic valve of any example herein, particularly example 29 or 30, wherein the internal surface of the inner skirt comprises a porous internal surface.

Example 32. The prosthetic valve of any example herein, particularly any one of examples 26 to 31, wherein the strip and the inner skirt are made of different materials.

Example 33. The prosthetic valve of any example herein, particularly any one of examples 26 to 32, wherein the inner skirt comprises at least one or more of a polytetrafluoroethylene, an ultra-high molecular weight polyethylene, or a coated thermoplastic polyurethane.

Example 34. The prosthetic valve of any example herein, particularly any one of examples 26 to 33, wherein the strip is coupled to the inner skirt by bonding or stitching.

Example 35. The prosthetic valve of any example herein, particularly any one of examples 26 to 34, wherein the inner skirt is coupled to the frame by suturing.

Example 36. The prosthetic valve of any example herein, particularly any one of examples 26 to 35, wherein the texture of the strip internal surface is different than the texture of the internal surface of the inner skirt.

Example 37. The prosthetic valve of any example herein, particularly example 29 or 36, wherein the internal surface of the inner skirt is smoother than the strip internal surface.

Example 38. The prosthetic valve of any example herein, particularly example 29 or 36, wherein both the strip internal surface and the internal surface of the inner skirt comprise pores, and wherein the pores of the internal surface of the inner skirt are narrower than the pores of the strip internal surface.

Example 39. The prosthetic valve of any example herein, particularly any one of examples 1 to 38, further comprising an outer skirt coupled to an outer surface of the frame.

Example 40. The prosthetic valve of any example herein, particularly example 29 or 39, wherein the outer skirt is coupled to the frame by suturing.

Example 41. The prosthetic valve of any example herein, particularly example 29 or 39 or 40, wherein the outer skirt is configured to allow tissue ingrowth.

Example 42. The prosthetic valve of any example herein, particularly example 29 or 41, wherein the outer skirt comprises yarns extending radially outward.

Example 43. The prosthetic valve of any example herein, particularly example 29 or 41, wherein the outer skirt comprises polyethylene terephthalate.

Example 44. The prosthetic valve of any example herein, particularly example 29 or 41, wherein the outer skirt comprises a knit pattern formed along an external surface thereof.

Example 45. The prosthetic valve of any example herein, particularly example 29 or 41, wherein the outer skirt comprises a porous texture.

Example 46. The prosthetic valve of any example herein, particularly any one of examples 26 to 35, wherein the outer skirt is a bioresorbable outer skirt.

Example 47. A prosthetic valve, comprising:
a frame movable between a radially compressed state and a radially expanded state;
an inner skirt coupled to an inner surface of the frame;
a strip coupled to an internal surface of the inner skirt and comprising a strip internal surface; and
a plurality of leaflets configured to regulate flow through the prosthetic valve, the plurality of leaflets coupled to the strip internal surface;
wherein the inner skirt is configured to allow formation of neointimal tissue that does not exceed an average thickness threshold; and
wherein the strip is configured to encourage adherence of tissue of any thickness to the strip internal surface.

Example 48. The prosthetic valve of any example herein, particularly example 47, wherein the average thickness threshold is 200 μm.

Example 49. The prosthetic valve of any example herein, particularly example 47 or 48, wherein the strip defines a strip height which is less than a height of the inner skirt.

Example 50. The prosthetic valve of any example herein, particularly example 49, wherein the strip height is non-uniform along a circumference of the strip.

Example 51. The prosthetic valve of any example herein, particularly any one of examples 47 to 50, wherein the strip and the inner skirt are made of different materials.

Example 52. The prosthetic valve of any example herein, particularly any one of examples 47 to 51, wherein the texture of the strip internal surface is different than the texture of the internal surface of the inner skirt.

Example 53. The prosthetic valve of any example herein, particularly example 52, wherein the internal surface of the inner skirt is smoother than the strip internal surface.

Example 54. The prosthetic valve of any example herein, particularly any one of examples 47 to 53, wherein the strip comprises yarns extending radially inward.

Example 55. The prosthetic valve of any example herein, particularly any one of examples 47 to 53, wherein the strip comprises polyethylene terephthalate.

Example 56. The prosthetic valve of any example herein, particularly any one of examples 47 to 53, wherein the strip comprises a knit pattern formed along the strip internal surface.

Example 57. The prosthetic valve of any example herein, particularly any one of examples 47 to 53, wherein the strip comprises a porous texture.

Example 58. The prosthetic valve of any example herein, particularly any one of examples 47 to 57, wherein the inner skirt comprises at least one or more of a polytetrafluoroethylene, an ultra-high molecular weight polyethylene, or a coated thermoplastic polyurethane.

Example 59. The prosthetic valve of any example herein, particularly any one of examples 47 to 58, wherein the inner skirt comprises a porous texture.

Example 60. The prosthetic valve of any example herein, particularly example 57, wherein the inner skirt comprises a porous texture, and wherein pores of the inner skirt are narrower than pores of the strip.

Example 61. The prosthetic valve of any example herein, particularly any one of examples 47 to 60, wherein each leaflet of the plurality of leaflets is coupled to the strip along a cusp edge thereof, wherein each cusp edge defines a cusp edge midpoint, and wherein the strip comprises a proximal portion extending from the cusp edge to a proximal edge of the strip along a proximal portion height.

Example 62. The prosthetic valve of any example herein, particularly example 61, wherein the proximal portion height at a circumferential position of the cusp edge midpoint is equal to or less than 20% of a radius of the prosthetic valve in the radially expanded state.

Example 63. The prosthetic valve of any example herein, particularly example 62, wherein the proximal portion height at the circumferential position of the cusp edge midpoint is equal to or less than 10% of the radius of the prosthetic valve in the radially expanded state.

Example 64. The prosthetic valve of any example herein, particularly any one of examples 47 to 60, wherein the proximal portion height is non-uniform along the circumference of the strip, defining a maximal proximal portion height at the circumferential position of the cusp edge midpoint.

Example 65. The prosthetic valve of any example herein, particularly any one of examples 61 to 64, wherein the strip further comprises a distal portion extending from the cusp edge to a distal edge of the strip along a distal portion height.

Example 66. The prosthetic valve of any example herein, particularly example 65, wherein the proximal portion height is greater than the distal portion height at the circumferential position of the cusp edge midpoint.

Example 67. The prosthetic valve of any example herein, particularly example 66, wherein the proximal portion height is at least two times greater than the distal portion height at the circumferential position of the cusp edge midpoint.

Example 68. The prosthetic valve of any example herein, particularly any one of examples 65 to 67, wherein the distal portion height is non-uniform along the circumference of the strip, defining a maximal distal portion height at the circumferential position of the cusp edge midpoint.

Example 69. The prosthetic valve of any example herein, particularly any one of examples 47 to 68, further comprising an outer skirt coupled to an outer surface of the frame.

Example 70. The prosthetic valve of any example herein, particularly example 69, wherein the outer skirt is configured to allow tissue ingrowth.

Example 71. The prosthetic valve of any example herein, particularly example 70, wherein the outer skirt comprises yarns extending radially outward.

Example 72. The prosthetic valve of any example herein, particularly example 70, wherein the outer skirt comprises polyethylene terephthalate.

Example 73. The prosthetic valve of any example herein, particularly example 70, wherein the outer skirt comprises a knit pattern formed along an external surface thereof.

Example 74. The prosthetic valve of any example herein, particularly example 70, wherein the outer skirt comprises a porous texture.

Example 75. The prosthetic valve of any example herein, particularly any one of examples 70 to 74, wherein the outer skirt is a bioresorbable outer skirt.

Example 76. The prosthetic valve of any example herein, particularly any one of examples 47 to 75, wherein the plurality of leaflets comprises three leaflets.

Example 77. The prosthetic valve of any example herein, particularly any one of examples 47 to 76, wherein the plurality of leaflets are made from biological material.

Example 78. The prosthetic valve of any example herein, particularly example 77, wherein the biological material comprises pericardium.

Example 79. The prosthetic valve of any example herein, particularly any one of examples 47 to 78, wherein the strip is configured to prevent spontaneous detachment of tissue adhered thereto for at least a predetermined period of time.

Example 80. The prosthetic valve of any example herein, particularly example 79, wherein the predetermined period of time is at least one month.

Example 81. The prosthetic valve of any example herein, particularly example 79, wherein the predetermined period of time is at least six months.

Example 82. The prosthetic valve of any example herein, particularly example 79, wherein the predetermined period of time is at least one year.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve comprising:
a frame movable between a radially compressed state and a radially expanded state;
a strip circumferentially disposed about an inner surface of the frame, the strip comprising a strip internal surface facing a central axis of the prosthetic valve, wherein the strip is configured to allow tissue adherence to the strip internal surface; and
a valvular structure coupled to the strip and comprising a plurality of leaflets extending inward from the strip internal surface.

2. The prosthetic valve of claim 1, wherein the valvular structure is coupled to the strip along a scalloped line, and wherein the strip comprises at least one inflow end portion and at least one outflow end portion.

3. The prosthetic valve of claim 2, wherein the strip comprises a proximal portion extending between the scalloped line and a proximal edge of the strip, wherein the proximal portion defines a proximal portion height.

4. The prosthetic valve of claim 3, wherein the proximal portion height at the at least one inflow end portion is equal to or less than 20% of a radius of the prosthetic valve in the radially expanded state.

5. The prosthetic valve of claim 3, wherein the proximal portion height is non-uniform between the at least one inflow end portion and the at least one outflow end portion.

6. The prosthetic valve of claim 5, wherein the proximal portion height at the at least one outflow end portion is smaller than the proximal portion height at the at least one inflow end portion.

7. The prosthetic valve of claim 2, wherein the strip further comprises a distal portion extending between the scalloped line and a distal edge of the strip, wherein the distal portion defines a distal portion height.

8. The prosthetic valve of claim 7, wherein the proximal portion height is greater than the distal portion height at the at least one inflow end portion.

9. The prosthetic valve of claim 8, wherein the proximal portion height is at least twice as great as the distal portion height at the at least one inflow end portion.

10. The prosthetic valve of claim 1, wherein the strip comprises yarns extending radially inward.

11. The prosthetic valve of claim 1, wherein the strip comprises a porous texture.

12. The prosthetic valve of claim 1, wherein the strip is configured to allow adherence of tissue exceeding an average thickness of 200 μm to the strip internal surface.

13. A prosthetic valve comprising:
a frame movable between a radially compressed state and a radially expanded state;
an inner skirt coupled to an inner surface of the frame;
a strip coupled to an internal surface of the inner skirt and comprising a strip internal surface; and
a plurality of leaflets configured to regulate flow through the prosthetic valve, the plurality of leaflets coupled to the strip internal surface;
wherein the inner skirt is configured to allow formation of neointimal tissue that does not exceed an average thickness threshold; and
wherein the strip is configured to encourage adherence of tissue of any thickness to the strip internal surface.

14. The prosthetic valve of claim 13, wherein the average thickness threshold is 200 μm.

15. The prosthetic valve of claim 13, wherein the strip defines a strip height which is less than a height of the inner skirt.

16. The prosthetic valve of claim 13, wherein the strip and the inner skirt are made of different materials.

17. The prosthetic valve of claim 13, wherein the texture of the strip internal surface is different than the texture of the internal surface of the inner skirt.

18. The prosthetic valve of claim 17, wherein the internal surface of the inner skirt is smoother than the strip internal surface.

19. The prosthetic valve of claim 13, wherein the strip comprises a porous texture.

20. The prosthetic valve of claim 19, wherein the inner skirt comprises a porous texture, and wherein pores of the inner skirt are narrower than pores of the strip.
